(19) United States
(12) Patent Application Publication
(10) Pub. No.: US 2026/0034292 A1
(43) Pub. Date: Feb. 5, 2026

(54) ASPIRATION DEVICES AND RELATED METHODS

(71) Applicant: Boston Scientific Scimed, Inc., Maple Grove, MN (US)

(72) Inventors: Abigail Christa GALLAGHER, Attleboro Falls, MA (US); Steven T. CARLSON, Minneapolis, MN (US); Rodolfo FINOCCHI, Ashland, MA (US); Niraj Prasad RAUNIYAR, Plymouth, MN (US); Michael S.H. CHU, Brookline, MA (US); Jessica WILLIAMS, Marlborough, MA (US)

(73) Assignee: Boston Scientific Scimed, Inc., Maple Grove, MN (US)

(21) Appl. No.: 19/285,172

(22) Filed: Jul. 30, 2025

Related U.S. Application Data
(60) Provisional application No. 63/679,477, filed on Aug. 5, 2024.

(51) Int. Cl. A61M 1/00; A61M 39/22

(52) U.S. Cl. CPC A61M 1/743; A61M 1/76; A61M 39/22; A61M 2205/0238

(57) ABSTRACT

Medical devices, systems, and related methods useful for providing suction or irrigation to body lumens and/or other body passageways during a medical procedure are described. The medical device may include a handle and a sheath coupled to and in fluid communication with the handle. The handle also may include a hub defining a lumen and a proximal opening, a suction port in fluid communication with the lumen, and a cover proximate an aperture of the suction port. The cover may be movable, e.g., pivotable or slidable, relative to the suction port to cover and uncover the aperture.

### CROSS-REFERENCE TO RELATED APPLICATION(S)

This application claims the benefit of priority to U.S. Provisional Application No. 63/679,477, filed on Aug. 5, 2024, which is incorporated by reference herein in its entirety.

### TECHNICAL FIELD

Aspects of the present disclosure relate generally to medical devices such as aspiration devices and related systems and methods. More specifically, the present disclosure relates to medical systems and devices with suction channels and ports, e.g., to facilitate aspiration during medical procedures.

### BACKGROUND

During a medical procedure, medical professionals often use medical devices coupled to a vacuum source to provide suction or irrigation during the medical procedure. However, the vacuum source is typically in an on state or off state, and it would be useful to have enhanced control over a resulting vacuum pressure applied to the medical device during the medical procedure.

### SUMMARY

The present disclosure relates to, among other things, medical devices and systems for providing suction or irrigation to body lumens and/or other body passageways during medical procedures, and related methods of use. Each aspect disclosed herein may include one or more features described in connection with any other disclosed aspect.

According to some aspects, the present disclosure includes a medical device including a handle and a sheath integral with or coupled to the handle. The handle may include a hub defining a lumen and a proximal opening, a suction port in fluid communication with the lumen and configured to be connected to a vacuum source, wherein the suction port includes an aperture, and a cover proximate the aperture. The sheath may define a channel in fluid communication with the lumen of the hub. The cover may be pivotable relative to a longitudinal axis of the suction port to cover and uncover the aperture, e.g., to control pressure through the lumen and the channel of the medical device.

According to some aspects, the cover may be coupled to the suction port by a hinge. For example, the cover may include a first end coupled to a hinge and a second end comprising an engagement element complementary to an engagement element of the hub or the suction port. The engagement element of the cover may include at least one of a latch, a hook, a pin, a magnet, or a combination thereof. In some examples, the engagement element of the cover may include a recess complementary to a projection of the suction port proximate a perimeter of the aperture. In some examples, the engagement element of the cover may include a tab complementary to a hook of the hub. Optionally, an inner surface of the cover may include a sealing element. In a closed position of the cover, the cover may extend at least partially through the aperture. According to some aspects, the cover may include a first portion and a second portion, each of the first portion and the second portion being pivotable relative to the suction port. A longitudinal axis of the suction port may be transverse to a longitudinal axis of the lumen of the hub. Additionally or alternatively, the hub may include a Tuohy Borst valve that defines the proximal opening. In some examples, the handle may be coupled to a tether, e.g., an elastic tether. Optionally, the hub may include a hydrophilic coating around the proximal opening. According to some aspects, the hub may include a hydrophilic ring that tapers inward to the proximal opening. At least a portion of an outer surface of the handle may include a rubber material, e.g., as an attachment.

The present disclosure also includes a medical device comprising a handle including a hub defining a lumen and a proximal opening, a suction port in fluid communication with the lumen and configured to be connected to a vacuum source, and a cover proximate the at least one aperture, wherein the suction port includes at least one aperture. The medical device also may include a sheath integral with or coupled to the handle, the sheath defining a channel in fluid communication with the lumen of the hub. The cover may be movable, e.g., slidable, over the at least one aperture to cover and uncover the at least one aperture. In some examples, the at least one aperture includes a plurality of apertures arranged in a pattern, and/or the at least one aperture has a triangular or teardrop shape. The aperture(s) may include at least three apertures, for example. The at least one aperture may have a triangular or teardrop shape. In some examples, a width of the at least one aperture increases in a direction towards the hub or increases in a direction away from the hub.

The present disclosure also includes a medical device comprising a handle including a hub defining a lumen and a proximal opening, a suction port in fluid communication with the lumen and configured to be connected to a vacuum source, and a cover proximate the aperture, wherein the suction port includes an aperture. The medical device may further comprise a sheath coupled to or integral with the handle, the sheath defining a channel in fluid communication with the lumen of the hub. Optionally, the medical device may include a tether, e.g., an elastic tether extending from a first end to a second end, e.g., the first end being coupled to the handle.

The present disclosure also includes medical systems comprising any of the medical devices discussed above and/or elsewhere herein, the medical system further comprising a medical instrument that includes a shaft extending through the proximal opening and slidable along the lumen of the hub into the channel of the sheath. When the medical system includes a tether, the second end of the tether may be coupled to the shaft of the medical instrument.

### BRIEF DESCRIPTION OF THE FIGURES

The accompanying drawings, which are incorporated in and constitute a part of this application, illustrate aspects of this disclosure and together with the description, serve to explain the principles of this disclosure. The drawings show exemplary medical devices useful for providing and controlling suction during a medical procedure.

FIGS. 1A-1B depict an exemplary medical device that includes a pivotable cover, according to some aspects of the present disclosure.

FIGS. 2A-2B and 3A-3B depict additional exemplary medical devices that include pivotable covers according to some aspects of the present disclosure.

FIGS. 4A-4B depict another exemplary medical device that includes a slidable cover according to some aspects of the present disclosure.

FIGS. 5A-5B depict another exemplary medical device that includes a pivotable cover and retention features according to some aspects of the present disclosure.

FIG. 6 depicts an exemplary medical system including a medical device and a shaft of a medical instrument coupled to the medical device, according to some aspects of the present disclosure.

FIG. 7 depicts an exemplary medical device that includes a valve according to some aspects of the present disclosure.

FIG. 8 depicts another exemplary medical device that includes a slidable cover and a plurality of apertures according to some aspects of the present disclosure.

FIGS. 9 and 10 depict exemplary seals for medical devices according to some aspects of the present disclosure.

FIG. 11 depicts another exemplary medical device with a slidable cover according to some aspects of the present disclosure.

FIG. 12 depicts another exemplary medical device that includes a slidable cover according to some aspects of the present disclosure.

FIG. 13 depicts another exemplary medical device that includes a slidable cover and a plurality of apertures according to some aspects of the present disclosure.

### DETAILED DESCRIPTION

Both the foregoing general description and the following detailed description are exemplary and explanatory only and are not restrictive of the features, as claimed. As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range. The terms “proximal” and “distal” are used herein to refer to the relative positions of the components of exemplary medical devices. As used herein, “proximal” refers to a position relatively closer to the exterior of the body or closer to an operator using the medical device (see proximal “P” and distal “D” directional arrows in the figures). In contrast, “distal” refers to a position relatively further away from the operator using the medical device, or closer to the interior of the body.

Aspects of the present disclosure are described herein with reference to exemplary medical devices, systems, and methods that may be useful for facilitating suction and/or irrigation during medical procedures. For example, the medical device may be an aspiration device. In some examples, the medical device includes a handle and a sheath configured to slidably receive a shaft (e.g., a shaft of a scope or other medical instrument), the handle including a hub and a suction port in fluid communication with the hub and the sheath. For example, the medical device may be configured to receive a medical instrument, e.g., via a proximal opening of the hub. The medical device may extend through the hub and the sheath, the medical instrument used to deliver irrigation at or proximate a target site of a subject while the medical device is used to apply suction at or proximate the target site. The medical device may be configured to allow a user to adjust suction pressure with the same hand used to grasp the handle of the medical device. For example, the user may employ features of the suction port to cover and uncover one or more apertures of the suction port.

The sheath may define a channel. When the medical device receives the shaft of a medical instrument, the channel may radially surround the shaft of the medical instrument to apply suction, e.g., to remove fluid(s) and/or other materials from the target site of a subject, via the suction port during a medical procedure. The suction port may include at least one aperture and a cover proximate the aperture. In some examples, the handle may include engagement elements to assist in selectively covering and uncovering the aperture with the cover. Engagement elements suitable for the present disclosure may include, but are not limited to, snap-fit, grooves, recesses, projections, threads, latches, locking pins, magnets, and friction fit. The handle may be configured to control suction pressure in the suction port during medical procedures, such that covering and uncovering the aperture allows the user to adjust and control magnitude of suction pressure therein.

References to a particular type of procedure, such as a urology procedure; a medical device, such as an aspiration device; and/or an organ, such as a kidney, are provided for convenience and not intended to limit this disclosure. Accordingly, analogous medical devices or systems may utilize one or more aspects of exemplary medical devices, systems, and methods described herein.

Reference is now made in detail to examples to help illustrate aspects of the present disclosure through the accompanying drawings. Wherever possible, the same reference numbers will be used throughout the drawings to refer to the same or like parts.

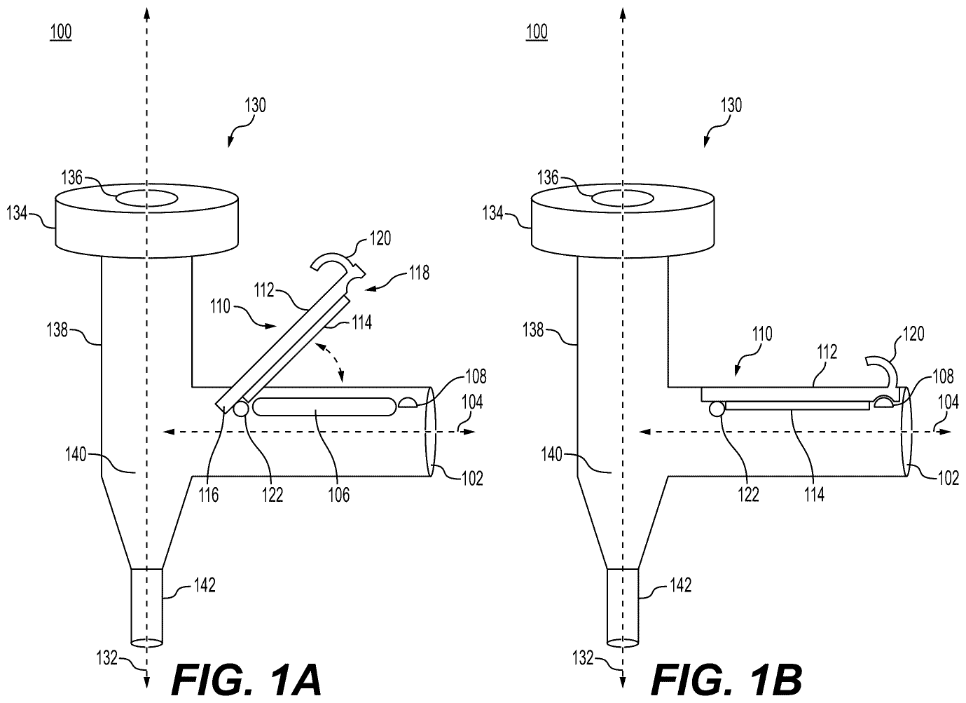

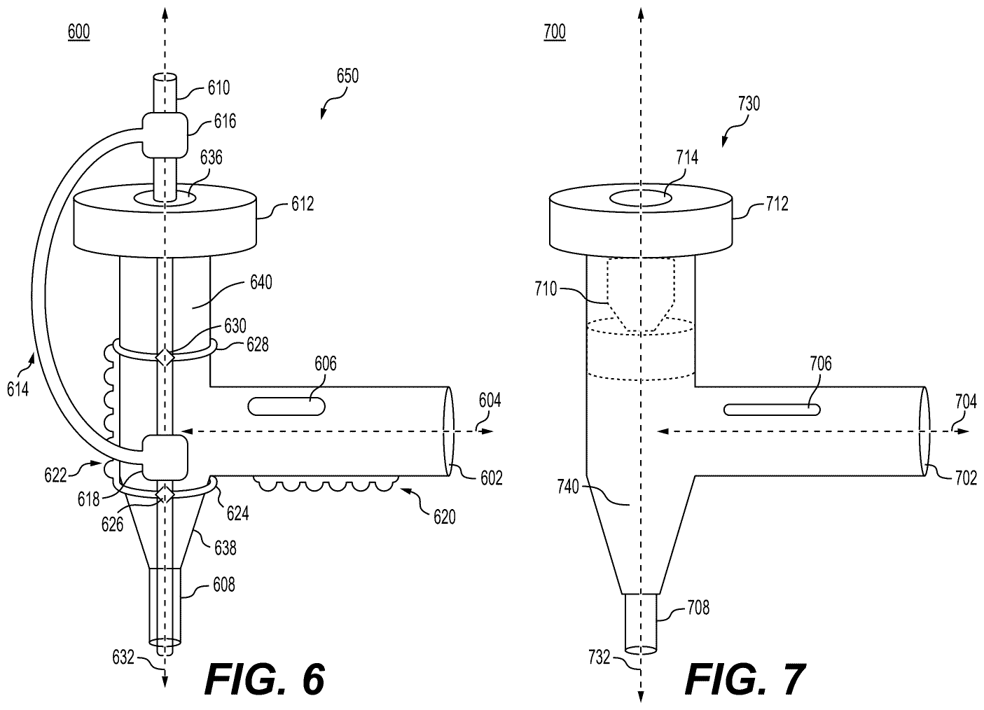

FIGS. 1A-1B illustrate an exemplary medical device 100 according to some aspects of this disclosure, medical device 100 including a sheath 142 and a handle 130. The medical device 100 may be configured to allow a user (e.g., medical professional) to deliver medical instruments to access internal areas of a subject's body, e.g., to perform medical diagnoses and/or treatments on the subject. The handle 130 may be coupled to or integral with the sheath 142, such that the handle 130 is in fluid communication with a channel of the sheath 142. The handle 130 may include a hub 138 with a proximal portion 134 defining a proximal opening 136 to slidably receive a medical instrument such as a scope (e.g., ureteroscope, endoscope, hysteroscope, bronchoscope, cystoscope, or other scope or similar device) into a lumen 140 defined by the handle 130. The medical instrument may be slidable along the lumen 140 of the handle 130 into the channel of the sheath 142 (see, e.g., FIG. 6 discussed below). In some examples, the medical instrument may be useful to deliver irrigation to a subject and/or provide tools to a target site of the subject. The proximal opening 136 may include a seal configured to maintain a position of a medical instrument relative to the medical device 100 and form a substantially airtight and/or watertight seal around the medical instrument.

The handle 130 includes a suction port 102 in fluid communication with the lumen 140 of hub 138. The suction port 102 may extend along a longitudinal axis 104 transverse to a longitudinal axis 132 of the hub 138. For the example shown, the longitudinal axis 104 of the suction port 102 may be orthogonal to the longitudinal axis 132 of the hub 138. The suction port 102 may be integral with or coupled to the hub 138. An end of the suction port 102 may be configured to be connected to a vacuum source to provide vacuum pressure through the suction port 102, the lumen 140 of the handle 130, and the channel of the sheath 142.

According to some aspects of the disclosure, the handle 130 may include one or more features that allow the user to control or adjust pressure through the suction port 102 when the suction port 102 is coupled to a vacuum source, e.g., during a medical procedure. In the example shown, the suction port 102 includes an aperture 106 (e.g., a vent) and a cover 110 proximate the aperture 106. The aperture 106 and the cover 110 may be proximate the hub 138 such that a user may be able to grasp the hub 138 and control movement of the cover 110 to cover and uncover the aperture 106 with the same hand.

For example, the cover 110 may be movable relative to the aperture 106. In this example, the cover 110 is pivotable relative to the suction port 102 to cover and uncover the aperture 106, e.g., via one or more movable elements, such as one or more hinges pivotably attaching the cover 110 to the suction port 102 proximate the aperture 106. The handle 130 may be movable between a first position wherein the aperture 106 is uncovered (FIG. 1A) and a second position wherein the cover 110 fully covers the aperture 106 (FIG. 1B). The medical device 100 may include a plurality of positions between the first and second positions wherein the aperture 106 is partially covered. As shown in FIGS. 1A-1B, the cover 110 includes an outer surface 112 having a first end 116 coupled to the suction port 102 via a hinge 122, and a second end 118 opposite the first end 116. The cover 110 may pivot about the first end 116 via the hinge 122 to cover and uncover aperture 106. Optionally, the cover 110 may include a sealing element 114 coupled to or otherwise disposed on an inner surface opposite the outer surface 112. The sealing element 114 may include a deformable, compressible, or otherwise moldable material configured to provide an airtight or watertight seal against the aperture 106. For example, the sealing element 114 may include an O-ring or a rubber material capable of conforming to the perimeter of the aperture 106.

As mentioned above, the handle 130 may include engagement elements to assist in maintaining a position of the cover 110 relative to the aperture 106. In the example shown in FIGS. 1A-1B, the suction port 102 includes a projection 108 proximate to the perimeter of the aperture 106, and the cover 110 includes a recess positioned at the second end 118 complementary to the projection 108. The projection 108 may snap or lock into engagement with the recess, e.g., to retain the cover 110 during use. Optionally, the cover 110 may include one or more surface features facilitating grasp by a user. For example, the cover 110 includes an extension 120 on the outer surface 112, e.g., proximate the second end 118. The extension 120 may be grasped by the user's finger.

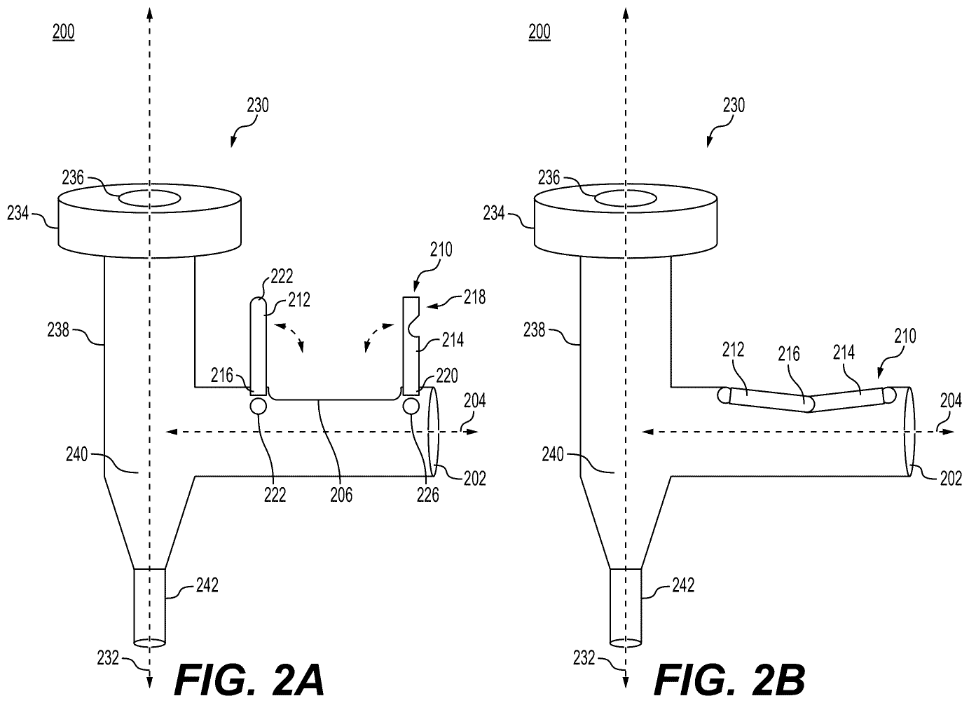

FIGS. 2A-2B illustrate another exemplary medical device 200 according to aspects of the disclosure. Unless otherwise specified, the medical device 200 may include any of the features of the medical device 100. As shown, the medical device 200 includes a handle 230 and a sheath 242 defining a channel in fluid communication with the handle 230. The handle 230 includes a hub 238 defining a lumen 240 and including a proximal portion 234 that defines a proximal opening 236.

The handle 230 also includes a suction port 202 in fluid communication with the lumen 240 of the hub 238, e.g., the suction port 202 extending along an axis 204 transverse to a longitudinal axis 232 of the hub 238. The suction port 202 includes an aperture 206 and a cover 210 proximate the aperture 206. The cover 210 may be movable relative to the aperture 206 to control pressure within the suction port 202 when coupled to a vacuum source. As shown, the cover 210 includes a first portion 212 and a second portion 214 each pivotable relative to the aperture 206. For example, the first portion 212 includes a first end 216 coupled to the suction port 202 via a first hinge 224 and the second portion 214 has a first end 220 coupled to the suction port 202 via a second hinge 226. A user may move the first and second portions 212, 214 to cover and uncover the aperture 206 to control pressure in the suction port 202 with the same hand grasping the handle 230. Thus, for example, the user may use one hand to control the medical device 200 (and adjust pressure through the medical device 200) and another hand to insert a shaft of a medical instrument (e.g., a scope) through the proximal opening 236 of the medical device 200.

The first portion 212 of cover 210 may be contoured to complement and engage the second portion 214 of cover 210. Optionally, the first portion 212 and/or second portion 214 may include engagement elements to assist in closing the aperture 206 when the first and second portions 212, 214 come together over the aperture 206. For example, the second portion 214 may include a groove or recess positioned at the second end 218 that is configured to receive the second end 222 of the first portion 212 or vice versa. The groove may complement the contour of the second end 222 of the first portion 212 to facilitate retention via snap-fit or compression fit.

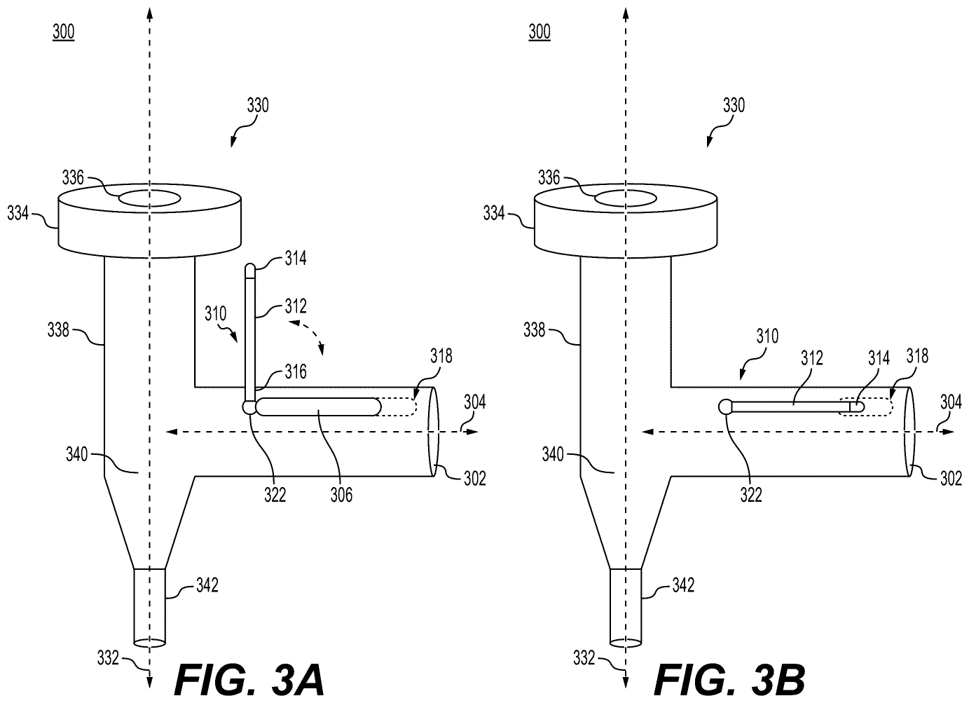

FIGS. 3A-3B illustrate another exemplary medical device 300, which may include any of the features of the medical device 100 and/or medical device 200 unless otherwise specified. As shown, medical device 300 includes a handle 330, and a sheath 342 defining a channel in fluid communication with the handle 330. The handle 330 may include a hub 338 defining a lumen 340 therein. The hub 338 may include a proximal portion 334 including a proximal opening 336.

The handle 330 also includes a suction port 302 extending along an axis 304 transverse to a longitudinal axis of the hub 338. The suction port 302 includes an aperture 306 and a cover 310 proximate the aperture 306 and movable relative to the aperture 306. For example, the cover 310 is pivotably coupled to the suction port 302 via a hinge 322 by grasping and moving a second end 314 of the cover 310 relative to a first end 316 of the cover. Optionally, an inner surface of the cover 310 opposite an outer surface 312 has a sealing element to assist in forming a seal with the aperture 306.

The suction port 302 may include a groove 318 along a portion of a perimeter of the aperture 306, the groove 318 configured to receive the second end 314 of the cover 310. In some examples, the second end 314 includes a tab configured to slide or snap into the groove 318. The cover 310 may include a flexible or bendable material, for example to allow the second end 314 to deform relative to the first end 316 as the cover 310 moves into, and out of, the groove 318.

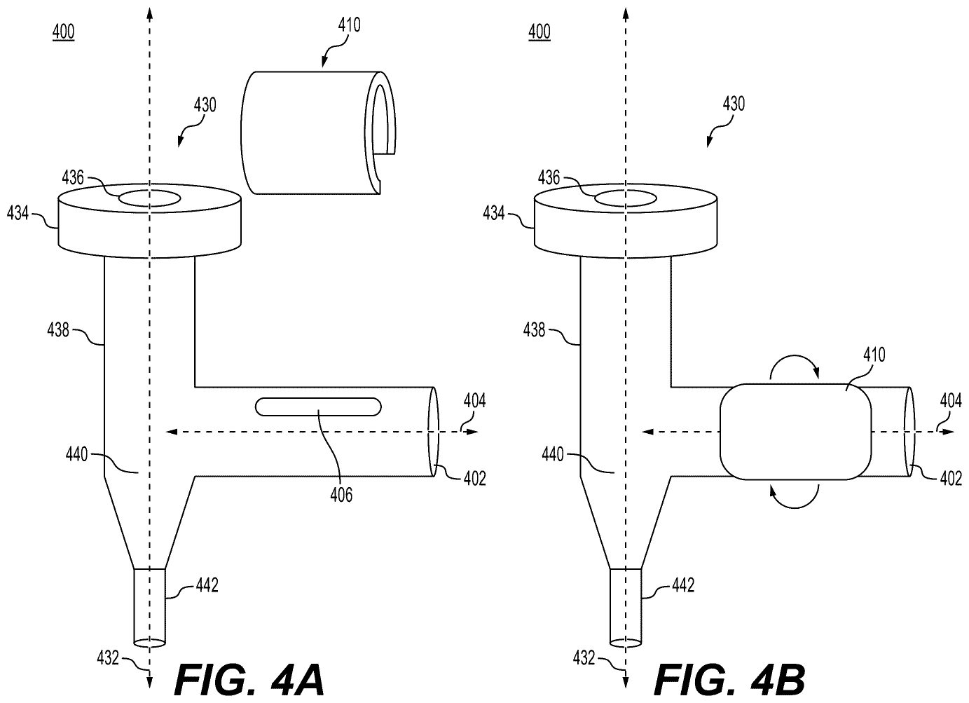

FIGS. 4A-4B illustrate another exemplary medical device 400 including a handle 430 and a sheath 442 defining a channel in communication with the handle 430. Unless otherwise specified, the medical device 400 may include any of the features of the medical devices 100, 200 and/or 300. The handle 430 includes a hub 438 defining a lumen 440 therein and a proximal portion 434 defining a proximal opening in communication with the lumen 440.

The handle 430 also includes a suction port 402 extending along an axis 404 transverse to a longitudinal axis of the hub 438. The suction port 302 includes an aperture 406 and a cover 410 proximate the aperture 406 to cover and uncover the aperture 406. FIG. 4A shows the cover 410 detached from the handle 430 in an exploded view for illustrative purposes; but the cover 410 may be secured to the handle 430 and rotatable relative to the suction port 402 in use as illustrated in FIG. 4B.

The cover 410 may be rotatable along an outer surface of the suction port 402 to cover and uncover the aperture 406. In some examples, the outer surface of the suction port 402 defines one or more grooves that receive the cover 410 to facilitate rotating the cover 410 relative to the suction port 402. A user may slide the cover 410 over the surface of the suction port 402 to gradually cover or uncover the aperture to increase or decrease pressure when the suction port 402 is coupled to a vacuum source. The user may grasp the handle 430 with the same hand used to move the cover 410.

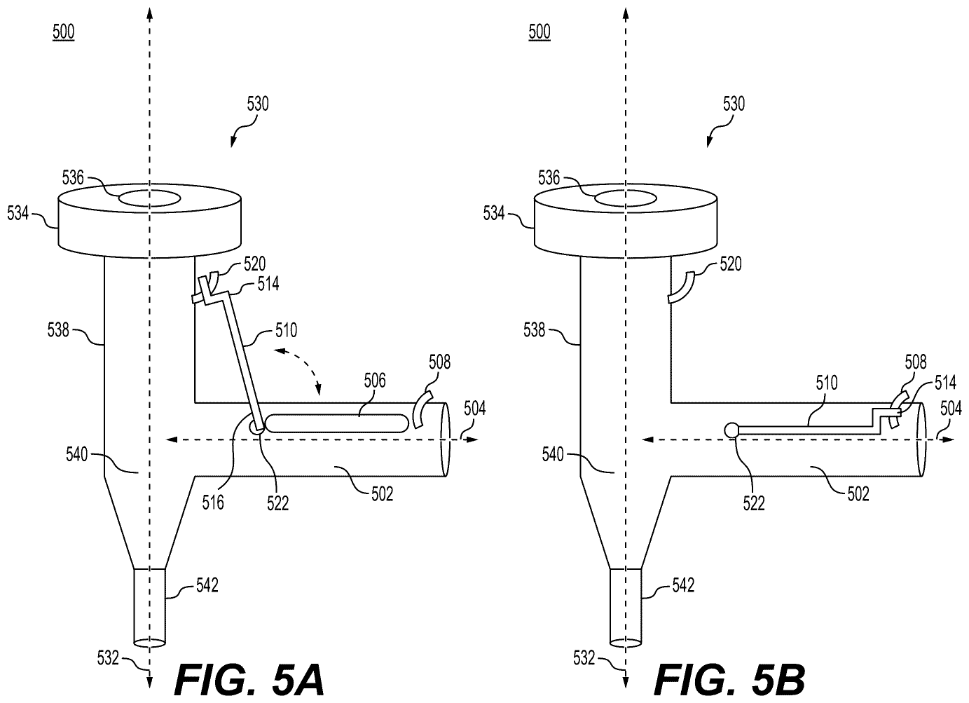

FIGS. 5A-5B illustrate another exemplary medical device 500 according to aspects of the present disclosure. Unless otherwise specified herein, the medical device 500 may include any of the features of medical devices 100, 200, 300, and/or 400. The medical device 500 includes a handle 530 and a sheath 542, the handle 530 including a hub 538 defining a lumen 540 in fluid communication with a channel defined by the sheath 542. The hub 538 may include a proximal portion 534 that defines a proximal opening capable of receiving the shaft of a medical instrument and described above and elsewhere herein (see, e.g., FIG. 6).

The handle 530 includes a suction port 502 extending along an axis 504 transverse to a longitudinal axis of the hub 538. The suction port 502 includes an aperture 506 and a cover 510 proximate the aperture 506 to cover and uncover the aperture 506 to control pressure when the suction port 502 is coupled to a vacuum source. The cover 510 includes a first end 516 pivotably coupled to the suction port 502 via a hinge 522, and a second end 518 that includes a tab 514 configured to engage a hook 520 of the hub 538 (e.g., to maintain a position when the aperture 506 is fully open) and a hook 508 of the suction port 502 (e.g., to maintain a position when the aperture 506 is fully closed).

FIG. 6 illustrates another exemplary medical device 600 according to aspects of the present disclosure. Unless otherwise specified herein, the medical device 600 may include any of the features of medical devices 100, 200, 300, 400, and/or 500. The medical device 600 includes a handle 650 and a sheath 608, the handle 650 including a hub 638 and a suction port 602. Also shown in FIG. 6 is a shaft 610 of a medical instrument inserted into the handle 650 via a proximal opening 636 of the hub 638. The medical instrument may include a scope or similar instrument that is insertable into the medical device 600.

The hub 638 defines a lumen extending along a longitudinal axis 632 and including a proximal portion 612 defining the proximal opening 636. The proximal portion 612 may include a seal, e.g., to assist in providing an airtight and/or watertight seal with the shaft 610. The suction port 602 may include any of the features of the suction ports 102, 202, 302, 402, and/or 502 discussed herein. For instance, the suction port 602 includes an aperture 606 that may be covered and uncovered by the hand of a user and/or by a cover as discussed above in connection to medical devices 100, 200, 300, 400, and/or 500. Thus, the user may cover and uncover the aperture 606 to adjust pressure through medical device 600 when the suction port 602 is coupled to a vacuum source.

The shaft 610 may be slidable along a channel defined by the sheath 608 (e.g., the sheath 608 being in fluid communication with a lumen 640 of the hub 638) such that a distal end of the shaft extends outside of the sheath. The medical device 600 includes a tether 614 extending from a first end 618 to a second end 616. The first end 618 may be coupled to the handle 650 and the second end 618 may be coupled to the shaft 610. One or both ends 616, 618 may be detachable from the handle 650 and/or the shaft 610. In some examples, the first end 618 is secured to an outer surface of the hub 638 and the second end 616 is coupled to, and detachable from, the shaft 610. Optionally, the tether 614 may include engagement elements to facilitate coupling to the handle 650 and/or the shaft 610. The tether 614 may be elastic, e.g., comprise an elastic material, e.g., to facilitate movement of the shaft 610 relative to the medical device 600 as the shaft 610 slides along the lumen of the handle 650 and the channel of the sheath 608.

The medical device 600 may include one or more surface features to assist a user in grasping the handle 650. For example, an outer surface of the handle 650 may include a rubber material 620 disposed on and/or around the hub 638 and/or the suction port 602 opposite the aperture 606. The rubber material 620 may provide a plurality of indents graspable by the user's hand. In this example, the rubber material 620 is secured to the outer surface of the suction port 602, however it should be understood that the surface features may be integral with the handle 650 or the rubber material may be included in other portions of the handle 650. The rubber material 622 includes a first portion 624 and a second portion 628 secured to different parts of the handle 650 grasped by a user's hand during use of the medical device 600. The handle 650 also includes a first clasp 626 on the first portion 624 and a second clasp 630 on the second portion 628 to assist in coupling the rubber material 622 to the handle 650.

FIG. 7 illustrates an exemplary medical device 700 that may include any of the features of medical devices 100, 200, 300, 400, 500, and/or 600 unless otherwise specified herein. The medical device 700 includes a handle 730 defining a lumen 740 and a sheath 708 defining a channel in fluid communication with the lumen of the handle 730. The handle 730 includes a hub 738 and a suction port 702 extending along an axis 704 transverse to a longitudinal axis 732 of the lumen 740. The suction port 702 includes an aperture 706 to adjust suction pressure when the suction port 702 is coupled to a vacuum source. The aperture 706 may be covered and uncovered by the hand of a user or by a cover as discussed in connection to medical devices 100, 200, 300, 400, and/or 500.

The hub 738 includes a valve 710 defining a proximal opening 714 for receiving a medical instrument. The valve 710 may be configured to inhibit or otherwise prevent backflow of fluid through the proximal opening 714. In some aspects, the valve 710 may include a Tuohy Borst valve. The valve 710 may include threads such that rotating the valve increases or decreases force against a shaft extending through the proximal opening 714 (see, e.g., FIG. 6) to control the strength of the seal against the shaft. In this way, rotating the valve 710 may control pressure through the medical device 700 when the suction port 702 is coupled to a vacuum source. Thus, for example, in some cases the medical device 700 does not include an aperture defined by the suction port 702, wherein a user may adjust pressure by rotating the valve 710.

Optionally, the handle 730 may include one or more markings to guide insertion of a shaft into the proximal opening 714, e.g., indicating different positions of the valve 710 to accommodate different sizes of scopes. In some examples, the proximal portion 712 includes a plurality of markings s circumferentially around the outer surface of the proximal portion 712, e.g., wherein each marking corresponds to a position of the valve 710 relative to the hub 738 and a corresponding size of the proximal opening 714. In some examples, the plurality of markings may correspond to a proximal opening size ranging from about 6 Fr (2.0 mm) to about 10 Fr (3.3 mm), such as from about 7.5 Fr (2.5 mm) to about 9.5 Fr (3.2 mm). Thus, for example, the valve 710 may be adjustable to accommodate a shaft having an outer diameter ranging from about 6 Fr to about 10 Fr, e.g., from about 7.5 Fr to about 9.5 Fr. A user may use the markings on the handle 730 to adjust the proximal opening 714 to receive a given shaft, and further rotate the valve 710 to increase or decrease the seal around the shaft.

FIG. 8 illustrates an exemplary medical device 800 including a handle 830 with a hub 838 and a suction port 802, and a sheath 842. The hub 838 may define a lumen in fluid communication with a channel defined by the sheath 842. The hub 838 may include a proximal portion 834 including a proximal opening 836 formed therein for receiving a shaft of a medical instrument. The medical device 800 may include any of the features of medical devices 100, 200, 300, 400, 500, 600, and/or 700 unless otherwise specified herein.

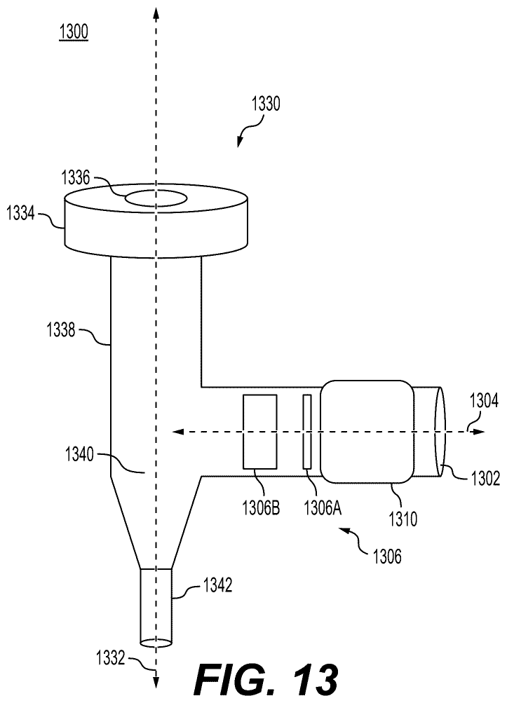

The suction port 802 defines a plurality of apertures 806, e.g., three apertures 806A, 806B, and 806C in this example. In other examples, the plurality of apertures 806 may include two apertures (e.g., 806A and 806B; see also FIG. 13) or may include four or more apertures 806. The apertures 806 may have the same size and/or shape or may be of different sizes and/or shape from each other (apertures of the same shape but different sizes shown in FIG. 8). The medical device 800 also includes a cover 810 proximate the apertures 806 and slidable along an outer surface of the suction port 802 to cover and uncover at least a portion or all of the apertures 806. In some examples, the cover 810 may include one or more surface features providing tactile and/or audio feedback corresponding to a position of the cover 810 relative to the apertures 806. For example, the cover 810 may include one or more surface features configured to make sound (e.g., “clicking”) as the cover 810 slides relative to apertures 806 between various positions along the suction port 802. In some examples, the apertures 806 may be arranged along the suction port 802 to increase in size in a direction towards the hub 838. In other examples, the apertures 806 may be arranged along the suction port 802 to decrease in size in the direction towards the hub 838.

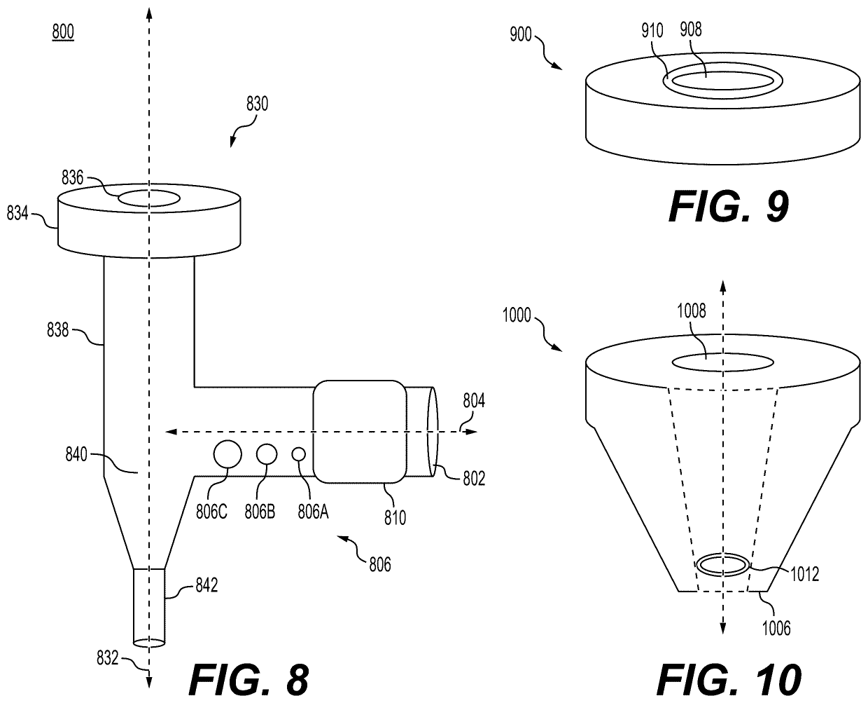

FIGS. 9 and 10 illustrate exemplary seals useful in connection to the medical devices herein, each seal defining a proximal opening for receiving a medical instrument. For example, FIG. 9 shows a seal 900 that includes a hydrophilic coating 910 around a proximal opening 908 of the seal 900, e.g., to alleviate friction with a medical instrument (e.g., shaft, scope, etc.). FIG. 10 shows a seal 1000 with a proximal opening 1008 and tapered profile towards a distal surface 1006. The seal 1000 also include a hydrophilic ring 1012.

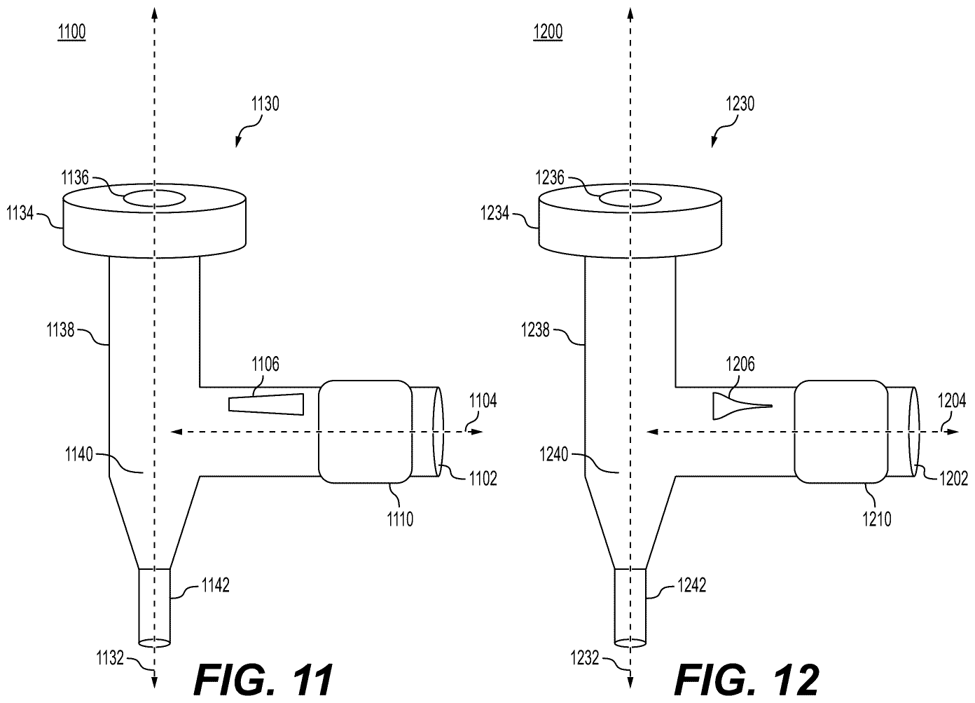

FIG. 11 illustrates another exemplary medical device 1100 that may include any of the features of the other medical devices discussed herein. As shown, the medical device 1100 includes a handle 1130 including a hub 1138 defining a lumen 1140 therein and a suction port 1102 extending along an axis 1104 transverse to the lumen of the hub 1138. The hub 1138 includes a proximal portion 1134 defining a proximal opening 1136 configured to receive a medical instrument therein. The medical device 1100 also includes a sheath 1142 defining a channel in fluid communication with the lumen 1140 of the handle 1130. The suction port 1102 includes an aperture 1106 having a tapered profile or perimeter along the suction port 1102. For example, aperture 1106 may have a generally trapezoidal shape. Other shapes of apertures with tapered profiles or perimeters like triangle and teardrop are contemplated herein (see, e.g., a generally teardrop shape in FIG. 12 discussed below). The profile of the aperture 1106 may narrow in a direction towards the hub 1138 as shown, or may widen in the direction towards the hub 1138. The medical device 1100 also includes a cover 1110 coupled to the suction port 1102 and slidable relative to the suction port 1102 to cover and uncover the aperture 1106. The tapered shape of the aperture 1106 may assist in an incremental adjustment of pressure as the cover 1110 is slid along the suction port 1102 to cover and uncover portions of the aperture 1106.

FIG. 12 illustrates another exemplary medical device 1200 that may include any of the features of the other medical devices discussed herein. As shown, the medical device 1200 includes a handle 1230 including a hub 1238 defining a lumen 1240 therein and a suction port 1202 extending along an axis 1204 transverse to the lumen 1240 of the hub 1238. The hub 1238 includes a proximal portion 1234 defining a proximal opening 1236 configured to receive a medical instrument therein. The medical device 1200 also includes a sheath 1242 defining a channel in fluid communication with the lumen 1240 of the handle 1230. Like medical device 1100, the suction port 1202 of medical device 1200 includes an aperture 1206 having a tapered profile or perimeter along the suction port 1202. For example, the aperture 1206 has a generally teardrop shape, wherein the profile or perimeter of the aperture 1206 widen in a direction towards the hub 1238. In some examples, aperture 1206 may have a triangular shape. The medical device 1200 also includes a cover 1210 coupled to the suction port 1202 and slidable relative to the suction port 1202 to cover and uncover the aperture 1206. As mentioned above, the tapered shape may assist in incremental adjustment of pressure, e.g., avoiding sudden changes in pressure as the cover 1210 is moved over the aperture 1206.

The shape and/or dimensions of the aperture(s) of the medical devices herein may be selected to provide for a controlled relationship between movement of a cover over the aperture(s) and pressure in the medical devices. For example, suction may increase or decrease non-linearly as the exposed cross-sectional area of the aperture(s) decreases or increases, respectively. This non-linear relationship between suction and exposed cross-sectional area may be counteracted by a tapered shape of the aperture(s). In some examples, the aperture(s) has a shape that tapers in only one direction, either increasing or decreasing along a longitudinal axis of the suction port (e.g., triangular and trapezoidal shapes). In some examples, the aperture(s) has a shape that tapers in more than one direction (e.g., teardrop or circular shapes). Shapes that taper in only one direction may provide better control over suction as compared to non-tapered shapes and shapes that taper in more than one direction. In some examples, the aperture(s) have a triangular shape, such as the shape of an isosceles triangle or an equilateral triangle.

Referring to FIGS. 11 and 12, for example, the exposed cross-sectional area of apertures 1106, 1206 may increase or decrease non-linearly as covers 1110, 1210 slide over apertures 1106, 1206, respectively. The tapered shape of apertures 1106, 1206 may provide more finely-tuned control of suction, e.g., avoiding sudden changes in pressure as covers 1110, 1210 slide over apertures 1106, 1206, respectively, as compared to an aperture having a non-tapered shape (e.g., a rectangle). In at least one example, the shape of aperture 1106 or aperture 1206 may be triangular, e.g., an isosceles or equilateral triangle (rather than trapezoidal or teardrop shape as illustrated).

FIG. 13 illustrates another exemplary medical device 1300 that may include any of the features of the other medical devices discussed herein. The medical device 1300 includes a handle 1330 including a hub 1338 defining a lumen 1340 therein and a suction port 1302 extending along an axis 1304 transverse to the lumen of the hub 1338. The hub 1338 includes a proximal portion 1334 defining a proximal opening 1336 configured to receive a medical instrument therein. The medical device 1300 also includes a sheath 1342 defining a channel in fluid communication with the lumen 1340 of the handle 1330. The suction port 1302 defines a plurality of apertures 1306, e.g., two apertures 1306A and 1306B of different size in this example. While both apertures 1306A, 1306B are shown having a rectangular shape, in other examples, the shape may be circular, oval, triangular, teardrop, etc. The medical device 1300 also includes a cover 1310 proximate the apertures 1306 and slidable along an outer surface of the suction port 1302 to cover and uncover at least a portion or all of the apertures 1306. The apertures 1306 may be arranged along the suction port 1302 to increase in size in a direction towards the hub 1338 as shown, or may be arranged along the suction port 1302 to increase in size in a direction away from the hub 1338.

While principles of this disclosure are described herein with reference to illustrative examples, it should be understood that the disclosure is not limited thereto. Those having ordinary skill in the art and access to the teachings provided herein will recognize additional modifications, embodiments, and substitution of equivalents all fall within the scope of the features described herein. Accordingly, the claimed features are not to be considered as limited by the foregoing description.

What is claimed is:

1. A medical device, comprising:
a handle including:
a hub defining a lumen and a proximal opening;
a suction port in fluid communication with the lumen and configured to be connected to a vacuum source, wherein the suction port includes an aperture; and
a cover proximate the aperture; and
a sheath coupled to the handle and defining a channel in fluid communication with the lumen of the hub,
wherein the cover is pivotable relative to a longitudinal axis of the suction port to cover and uncover the aperture.

2. The medical device of claim 1, wherein the cover is coupled to the suction port by a hinge.

3. The medical device of claim 1, wherein the cover includes a first end coupled to a hinge and a second end comprising an engagement element complementary to an engagement element of the hub or the suction port.

4. The medical device of claim 3, wherein the engagement element of the cover includes a recess complementary to a projection of the suction port proximate a perimeter of the aperture.

5. The medical device of claim 3, wherein the engagement element of the cover includes a tab complementary to a hook of the hub.

6. The medical device of claim 3, wherein the engagement element of the cover includes at least one of a latch, a hook, a pin, a magnet, or a combination thereof.

7. The medical device of claim 1, wherein a longitudinal axis of the suction port is transverse to a longitudinal axis of the lumen of the hub.

8. The medical device of claim 1, wherein an inner surface of the cover includes a sealing element.

9. The medical device of claim 1, wherein in a closed position of the cover, the cover extends at least partially through the aperture.

10. The medical device of claim 1, wherein the cover comprises a first portion and a second portion, each of the first portion and the second portion being pivotable relative to the suction port.

11. The medical device of claim 1, wherein the hub includes a Tuohy Borst valve that defines the proximal opening.

12. The medical device of claim 1, wherein at least a portion of an outer surface of the handle includes a rubber material.

13. The medical device of claim 1, wherein the handle is coupled to a tether.

14. The medical device of claim 1, wherein the hub includes a hydrophilic coating around the proximal opening.

15. The medical device of claim 1, wherein the hub includes a hydrophilic ring that tapers inward to the proximal opening.

16. A medical device, comprising:
a handle including:
a hub defining a lumen and a proximal opening;
a suction port in fluid communication with the lumen and configured to be connected to a vacuum source, wherein the suction port includes at least one aperture; and
a cover proximate the at least one aperture; and
a sheath coupled to the handle and defining a channel in fluid communication with the lumen of the hub,
wherein the cover is slidable over the at least one aperture to cover and uncover the at least one aperture; and
wherein the at least one aperture includes a plurality of apertures arranged in a pattern and/or the at least one aperture has a triangular or teardrop shape.

17. The medical device of claim 16, wherein the at least one aperture includes at least three apertures.

18. The medical device of claim 16, wherein the at least one aperture has a triangular or teardrop shape, a width of the at least one aperture increasing in a direction towards the hub.

19. A medical device, comprising:
a handle including:
a hub defining a lumen and a proximal opening;
a suction port in fluid communication with the lumen and configured to be connected to a vacuum source, the suction port including an aperture; and
a cover proximate the aperture,
a sheath coupled to the handle and defining a channel in fluid communication with the lumen of the hub; and
a tether extending from a first end coupled to the handle to a second end.

20. A medical system comprising the medical device of claim 19 and a medical instrument that includes a shaft extending through the proximal opening and slidable along the lumen of the hub into the channel of the sheath, wherein the second end of the tether is coupled to the shaft.
